(19) United States
(12) Patent Application Publication
(10) Pub. No.: US 2008/0261870 A1
(43) Pub. Date: Oct. 23, 2008

(54) Use of the Pro-Peptide Domain of Lysyl Oxidase as a Therapeutic Agent

(76) Inventors: Philip C. Trackman, Newton, MA (US); Amitha H. Palamakumbura, Winchester, MA (US); Gail E. Sonenshein, Brookline, MA (US); Sebastien Jeay, Brighton, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/585,651

(22) PCT Filed: Jan. 6, 2005
(86) PCT No.: PCT/US05/00631

§ 371 Date: Jul. 7, 2006

Related U.S. Application Data
(60) Provisional application No. 60/536,109, filed on Jan. 13, 2004.

(51) Int. Cl. A61K 38/17; C12Q 1/02; A61P 35/04

(52) U.S. Cl. 514/12; 435/29

(57) ABSTRACT

A therapeutic composition that includes an active portion of the lysyl oxidase pro-peptide in a pharmaceutically acceptable carrier substance and methods of using such a therapeutic composition are disclosed. The active agent does not have lysyl oxidase enzymatic activity. Preferably, the active polypeptide is active in inhibiting cell growth in soft agar and active in inhibiting tumor formation. In addition, the active polypeptide preferably comprises an active portion of the amino acid sequence given in SEQ ID NO.: 1 or SEQ ID NO.: 2, or conservative substitions thereof. Alternatively, the active polypeptide comprises a polypeptide comprising an active portion of an amino acid sequence selected from the group consisting of SEQ ID NOs.: 3-8, or conservative substitions thereof.

### CROSS REFERENCE TO RELATED APPLICATIONS

This application claims the benefit of U.S. Provisional Application No. 60/536,109, filed Jan. 13, 2004, the entire contents of which are hereby incorporated by reference herein.

### STATEMENT REGARDING FEDERALLY SPONSORED RESEARCH OR DEVELOPMENT

Part of the work leading to this invention was carried out with support from the United States Government under Grant Nos. DE 12425, CA 82742 and PO1-ES-11624-01 awarded by the National Institutes of Health and Grant No. DAMD 17-03-1-0452 awarded by the Department of the Army. Therefore, the U.S. Government has certain rights in this invention.

### BACKGROUND OF THE INVENTION

Lysyl oxidase catalyzes oxidative deamination of peptidyl lysine and hydroxylysine residues in collagens, and peptidyl lysine residues in elastin. The resulting peptidyl aldehydes spontaneously condense and undergo oxidation reactions to form the lysine derived covalent cross-links required for the normal structural integrity of the extracellular matrix (Kagan, 1986; Kagan et al., 1991; Kagan et al., 2003). Lysyl oxidase is synthesized as a 48-50 kDa pro-enzyme and secreted into the extracellular environment where it is then processed by proteolytic cleavage to a functional 30 kDa enzyme and an 18 kDa pro-peptide (Bedell-Hogan et al., 1992). The 30 kDa form of lysyl oxidase is an active enzyme whereas the 50 kDa pro-enzyme is enzymatically inactive (Trackman et al., 1992; Panchenko et al., 1996; Uzel et al., 2001). Procollagen C-proteinases are active in processing pro-lysyl oxidase (Panchenko et al., 1992; Uzel et al., 2001; Kessler et al., 1996).

Lysyl oxidase gene expression inhibits the transforming activity of ras and, hence, was named the “ras recision gene” (rrg) (Contente et al., 1990; Kenyon et al., 1991). Lysyl oxidase is down-regulated in ras-transformed cells and in many cancer cell lines. Reduced lysyl oxidase levels are also observed in human cancers (Contente et al., 1990; Kuivaniemi et al., 1986; Ren et al., 1998; Krzyzosiak et al., 1992; Hajnal et al., 1993; Hamalainen et al., 1995), whereas in spontaneous revertants or upon induced phenotypic reversion, higher, normal levels of lysyl oxidase are again seen (Contente et al., 1990; Hajnal et al., 1993). Conversely, stable phenotypic revertants of ras-transfected NIH 3T3 cells return to a transformed phenotype upon transfection with an anti-sense lysyl oxidase vector (Contente et al., 1990; Kenyon et al., 1991; Trackman et al., 1990). Anti-sense lysyl oxidase transfection triggers transformation of normal rat kidney fibroblasts (Giampuzzi et al., 2001). Thus, the lysyl oxidase gene has tumor suppressor activity, although the mechanism of this activity is unknown.

### BRIEF SUMMARY OF THE INVENTION

It has now been determined that the released pro-peptide is responsible for the ras-recision activity. After making the lysyl oxidase pro-peptide protein in E. coli by recombinant DNA technology, purifying the pro-peptide and confirming its protein sequence, we asked whether the pro-peptide influences the growth characteristics of c-H-ras transformed cells, first by cell cycle analyses and then by simple growth curves. We found that the pro-peptide both modulated the cell cycle and inhibited the growth of these cells. We then asked whether the pro-peptide inhibits the transformed phenotype by assessing its effect on the growth of ras-transformed cells in soft agar. Growth in soft agar is a hallmark of the transformed phenotype, and phenotypically normal cells are unable to grow and form colonies in soft agar. Results in replicate experiments show that the lysyl oxidase pro-peptide prevents the growth in soft agar of c-H-ras transformed cells. The mature active enzyme purified from bovine aorta was not able to inhibit growth in soft agar. Furthermore, breast cancer cells transformed by a different oncogene Her-2/neu, which signals by a pathway that overlaps c-Ha-ras, was also inhibited by the lysyl oxidase pro-peptide in the growth-in-soft-agar assay. However, cells transformed by a different oncogene (c-myc), which utilizes different signaling pathways, are not inhibited by the lysyl oxidase pro-peptide, demonstrating specificity and lack of toxicity of the pro-peptide.

As the lysyl oxidase pro-peptide inhibits ras-dependent cell transformation, this pro-peptide, or active portions thereof, would be useful as therapeutic agents, particularly anti-cancer therapeutic agents. Thus, the invention is directed to a therapeutic composition that includes an active portion of the lysyl oxidase pro-peptide in a pharmaceutically acceptable carrier substance and to methods of using such a therapeutic composition. The active agent does not have lysyl oxidase enzymatic activity. Preferably, the active polypeptide is active in inhibiting cell growth in soft agar and active in inhibiting tumor formation. In addition, the active polypeptide preferably comprises an active portion of the amino acid sequence given in SEQ ID NO.: 1 or SEQ ID NO.: 2, or conservative substitions thereof. Alternatively, the active polypeptide comprises a polypeptide comprising an active portion of an amino acid sequence selected from the group consisting of SEQ ID NOs.: 3-8, or conservative substitions thereof.

In another embodiment, the invention is directed to a method of treating a patient, comprising the steps of providing a patient suffering from cancer; and administering to the patient a therapeutically effective amount of the composition according the invention. Preferably, the patient suffers from a form of cancer dependent on ras signaling for cell transformation (e.g., colon, breast, lung or prostate cancer. In an alternative treatment method, the patient suffers from a disease or disorder that occurs via elevated ras-dependent signaling, such as a disease or disorder of the kidney, cardiovascular system and immune system, such as a bone disease, specifically an osteopenic condition such as osteoporosis.

Assays to detect the effectiveness of the lysyl oxidase pro-peptide in inhibiting, e.g., the growth of transformed cells can be used to determine active portions thereof. For example, using the soft agar assay described herein, or any cell culture assay, the activity of progressively smaller portions of the pro-peptide can be tested until the minimum sized active portion is determined.

Separate experiments carried out in normal differentiating osteoblast (bone cell) cultures show that the lysyl oxidase pro-peptide delays osteoblast differentiation, but interestingly appears to result ultimately in greater formation of a mineralized extracellular matrix. Thus, the therapeutic composition of the invention can also be used to treat osteopenia associated with diseases such as osteoporosis, or diabetic osteopenia, or other bone pathologies. Similarly, other disease conditions including kidney (Hendry and Sharpe, 2003), cardiovascular (Cvejic et al., 2000; Molkentin and Dorn, 2001), and immune system disorders (Cantrell, 2002; Schwartz, 2003; Wong et al., 2002), which occur via elevated ras-dependent signaling, seem likely to be improved by exposure to the lysyl oxidase pro-peptide.

### BRIEF DESCRIPTION OF THE SEVERAL VIEWS OF THE DRAWINGS

Other features and advantages of the invention will be apparent from the following description of the preferred embodiments thereof and from the claims, taken in conjunction with the accompanying drawings, in which:

FIGS. 1A and 1B. Lysyl oxidase pro-peptide, but not enzyme, inhibits growth of transformed cells; AS-3B cells (A) and RS485 cells (B). Cells were plated in 24-well plates (7,000 cells/well) pre-coated with 0 μg (), 0.2 μg (□), 1 μg (▴), 2 μg (◯), or 4 μg (★) recombinant rat lysyl oxidase pro-peptide/well. In A, cells were plated in addition on 4 μg mature 30 kDa lysyl oxidase (ρ). At the indicated times, cells were stained with crystal violet, quantitated by spectrophotometry at 590 nm, and growth curves obtained. Each data point is the average of values from 3 wells +/−SD. In (B) the inset is the same data directly plotted as absorbance +/−SD vs time to more clearly show the growth inhibitory effect of the lysyl oxidase pro-peptide;

FIGS. 2A and 2B. Lysyl oxidase pro-peptide inhibits growth of RS485 cells in soft agar. (A) Crystal violet stained colonies of RS485 cells, or myc-transformed M158 cells (myc) grown in soft agar in the presence of 30 kDa mature lysyl oxidase enzyme (+LO), recombinant rat lysyl oxidase pro-peptide (+LO pro-peptide), or vehicle control. (B) Colonies were counted in three independent fields, and values per field +/−SD expressed as percent of vehicle control; and

FIG. 3A is the amino acid sequence of human lysyl oxidase pro-peptide, from amino acid residues 22-168 (SEQ ID NO.: 1) (Mariani et al., 1992);

FIG. 3B is the amino acid sequence of mouse lysyl oxidase pro-peptide, from amino acid residues 22-162 (SEQ ID NO.: 2) (Mariani et al., 1992);

FIG. 3C shows alignment of the first conserved region of lysyl oxidase pro-peptide from human, mouse and rat, respectively (SEQ ID NOs.: 3-5) (http://blocks.fhcrc.org); and

FIG. 3D shows alignment of the second conserved region of lysyl oxidase pro-peptide from human, mouse and rat, respectively (SEQ ID NOs.: 6-8) (http://blocks.fhcrc.org).

### DETAILED DESCRIPTION OF THE INVENTION

It has now been determined that the ability of lysyl oxidase to revert the phenotype of ras-transformed fibroblasts depends substantially on the pro-peptide domain and not on lysyl oxidase enzyme activity. Since diminished lysyl oxidase expression in some way contributes to the transformed phenotype, it had generally been assumed that lysyl oxidase enzyme activity is related to the tumor suppressor activity of lysyl oxidase, and, therefore, that diminished lysyl oxidase activity promotes the transformed phenotype. However, β-aminopropionitrile (BAPN), the specific inhibitor of lysyl oxidase enzyme activity, did not prevent suramin-mediated reversion of the transformed phenotype, which is accompanied by increased lysyl oxidase expression. These findings were confirmed in the stable phenotypic revertant cell line PR4, that requires lysyl oxidase expression for normal phenotype maintenance; yet inhibition of lysyl oxidase activity with BAPN failed to re-transform these cells. Similarly, BAPN failed to block the ability of ectopic lysyl oxidase expression to prevent growth of ras-transformed fibroblasts in soft agar. The lack of effect of BAPN on lysyl oxidase-dependent phenotype control is interesting. Intracellular localization of mature lysyl oxidase has been shown to occur via normal extracellular processing of pro-lysyl oxidase, followed by uptake of mature lysyl oxidase (Nellaiappan et al., 2000). Given that BAPN is an irreversible inhibitor of lysyl oxidase (Tang et al., 1983), it follows that both extracellular and intracellular lysyl oxidase activity are susceptible to inhibition by BAPN. These findings, therefore suggest that neither extracellular nor intracellular lysyl oxidase activity contribute significantly to inhibiting the transformed cell phenotype.

The importance of lysyl oxidase expression in maintaining a normal cell phenotype in suramin treated RS485 cells was supported by anti-sense transfection studies. Most important, using recombinant lysyl oxidase pro-peptide, we demonstrated that the lysyl oxidase pro-peptide itself directly stimulates phenotypic reversion of ras-transformed cells, as judged by rate of proliferation and cell cycle colony formation in soft agar. This contrasts with the absence of an effect of the lysyl oxidase enzyme on both the growth rate of AS-3B cells and on colony formation in soft agar of RS485 cells. Thus, the independent activity of the lysyl oxidase pro-peptide will be therapeutically significant in the treatment of cancers in which ras-dependent pathways are abnormally active.

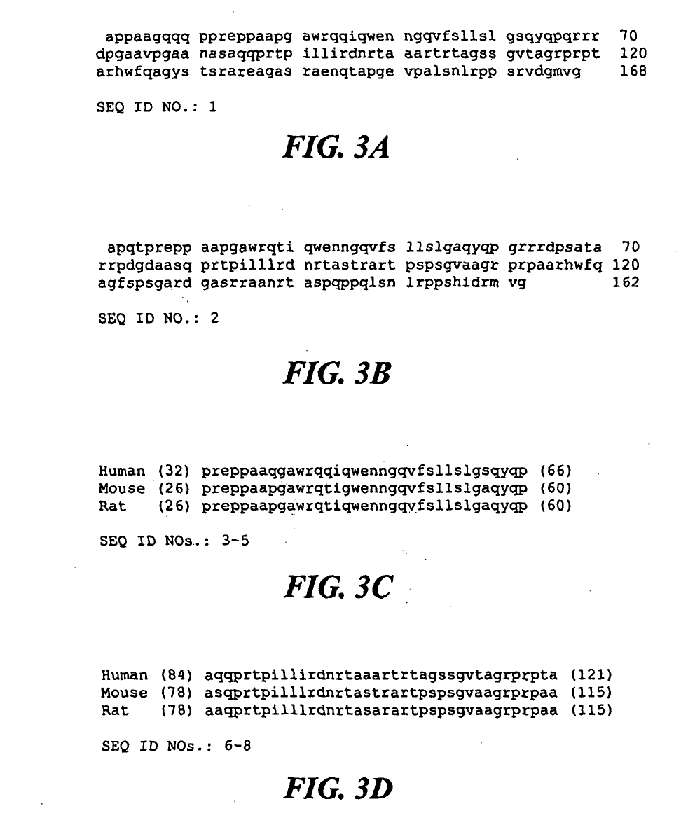

Structural features of the lysyl oxidase pro-peptide contribute to an understanding of its activity. The biosynthesis of lysyl oxidase occurs by secretion of a 50 kDa precursor, followed by extracellular proteolytic processing to form active 30 kDa lysyl oxidase and the 18-20 kDa pro-peptide (Trackman et al., 1992; Panchenko et al., 1996; Uzel et al., 2001). Unlike the anionic C-terminal region of pro-lysyl oxidase that becomes the active enzyme after processing (Panchenko et al., 1996; Uzel et al., 2001), the N-terminal pro-peptide region is rich in arginine and is cationic with a calculated pI of 12.5 for the mouse, rat, and human proteins. (See FIGS. 3A and 3B.) The highly basic character of the lysyl oxidase pro-peptide appears to facilitate its uptake by cells where it might exert its biological function, possibly entering cells even in the absence of a specific receptor. Cell membranes are permeable to arginine-rich basic proteins, and uptake of these basic proteins is mediated by heparin sulfate proteoglycans (Belting, 2003). The arginine-rich highly basic pro-peptide region of lysyl oxidase is less well conserved between species than the mature enzyme (Mariani et al., 1992), but it contains two blocks of 35 and 37 amino acids residues in length, respectively, that are nearly perfectly conserved among mouse, rat and human, and highly conserved in chicken lysyl oxidase. These regions are residues 26-60, and 78-115, respectively, in the mouse lysyl oxidase sequence. (See FIGS. 3C and 3D.) This high degree of similarity suggests that biological activities of the lysyl oxidase pro-peptide reside in these conserved sequences. Lysyl oxidase is a member of a multi-gene family, and it is notable that the sequence of the lysyl oxidase pro-peptide region is not well conserved among other lysyl oxidase family members, whereas the catalytic domains are well conserved. Lysyl oxidase itself, and not the lysyl oxidase-like genes, has been consistently identified in screens for tumor suppressors and is expressed at low levels in transformed cells and at higher levels in phenotypically normal cells (Contente et al., 1990; Ren et al., 1998). The finding of phenotype modulating activities occurring in regions of lysyl oxidase that are located in the unique pro-peptide domain may help to explain why lysyl oxidase itself is a tumor suppressor.

### Use

Colon, breast, lung, prostate cancers are frequently linked to overexpression or mutations in ras genes, or in pathways that depend on ras activity. Ras dysfunction is a determining factor of the pathology in many of these cancers, as well as other types of cancer. The therapeutic compositions of the invention comprising the lysyl oxidase pro-peptide will be therapeutically effective in treating forms of cancer that are dependent upon ras signaling for cell transformation. Furthermore, diseases that involve dysregulated ras-mediated induction of proliferation or inhibition of differentiation will also be susceptible to treatment with the pro-peptide.

For example, the lysyl oxidase pro-peptide inhibits osteoblast proliferation and slows bone cell differentiation. This inhibition may be a normal aspect of osteoblast cell biology and could contribute to normal bone formation and normal bone quality and integrity. The proper balance between factors that, respectively, stimulate or inhibit osteoblast differentiation contribute to normal bone formation and maintenance. Alterations in this balance lead to bone pathology. Thus, a role for abnormal or diminished levels of the lysyl oxidase pro-peptide contributing to bone diseases such as osteoporosis is likely. For example, lysyl oxidase dependent cross-linking is well documented to be diminished in osteoporosis, and diminished lysyl oxidase biosynthesis could contribute to this phenomenon. Thus, to the extent that the lysyl oxidase pro-peptide could normalize osteoblast cell biology, there is the potential that administration of this peptide to diseased bone tissues could help to normalize the structure of osteoporotic bone. This notion is applicable to other osteopenic conditions and bone diseases.

In general, therapeutic compositions according to the invention may be administered orally, topically, or parenterally, (e.g., intranasally, subcutaneously, intramuscularly, intravenously, or intra-arterially) by routine methods in pharmaceutically acceptable inert carrier substances. For example, the therapeutic compositions of the invention may be administered in a sustained release formulation using a biodegradable biocompatible polymer, or by on-site delivery using micelles, gels or liposomes. The therapeutic compositions can be administered, e.g., in a dosage of 0.25 μg/kg/day to 5 mg/kg/day. Optimal dosage and modes of administration can readily be determined by conventional protocols.

When the active compound is the unmodified lysyl oxidase pro-peptide, it cannot be given orally. The peptide or fragment derived from the pro-peptide will be administered by injection either systemically or locally, depending on results from pilot studies in animals. A peptide mimetic, when developed, may be given orally if proven effective.

A peptide mimetic can be designed as follows. First, molecular modeling studies based on the primary sequence of the lysyl oxidase pro-peptide are performed in order to predict the three dimensional structure. Particular attention will be paid to the structure of regions of the pro-peptide that are conserved and are likely to be functional. As structure/function studies progress, attention can shift to known functional sequences. Regions of the peptide whose structure can be predicted and are of interest will then be used as the basis for the design of non-peptidyl chemical structures that mimic the three dimensional features of the peptide region.

In addition gene therapy approaches can be considered. Specifically, a therapeutic strategy that results in production in patients (in vivo) of either the lysyl oxidase pro-peptide or an active portion of the lysyl oxidase pro-peptide could be beneficial. In this approach, an expression vector for the lysyl oxidase pro-peptide, or active portion of the pro-peptide is created such that high level production can be achieved following administration of the vector directly, or in the form of cells engineered to overexpress the pro-peptide active portion, to patients. Expression could be inducible by small molecule drugs such as tetracycline. Vectors could include, but are not limited to, adenovirus based technology.

It is anticipated that the therapeutic composition of the invention will be useful as a supplement to other chemotherapeutics administered as adjuvant chemotherapy therapy following surgical resection of tumors. For example the lysyl oxidase pro-peptide could be used in conjunction with adjuvant hormone replacement therapy or chemotherapy (anastrozole, tamoxifen, aromatiase inhibitors, cyclophosphamide, methotrexate, 5-fluorouracil, anthracycline or combinations) to treat breast cancer. Similarly, supplementation of current chemotherapy practice with pro-peptide or active fragment derived from the pro-peptide to treat other forms of cancer is envisioned.

As described earlier, the sequence of the numerous lysyl oxidase pro-peptides, e.g., human, mouse and rat, is known. Structure function studies as described herein will identify the active regions that mediate the anti-tumor activity of the lysyl oxidase pro-peptide and are useful in the composition of the invention, as described herein.

The following examples are presented to illustrate the advantages of the present invention and to assist one of ordinary skill in making and using the same. These examples are not intended in any way otherwise to limit the scope of the disclosure.

Effect of suramin on RS485 cell phenotype. Suramin is a polysulfonated naphthylurea, initially used in the treatment of trypanosomiasis and onchocerciasis (Sullivan et al., 1997). Its anti-cancer activity was later identified, and suramin has been introduced into clinical trials for various forms of cancer (La Rocca et al., 1990(a); La Rocca et al., 1990(b); Song et al., 2001; Small et al., 2000; Garcia-Schurmann et al., 1999). Suramin interrupts autocrine growth factor pathways by inhibiting the binding of growth factors to their receptors (Sullivan et al., 1997; Zanghi et al., 2000; Fukumoto et al., 2000; Mietz et al., 1998; Coffey et al., 1987). Treatment of c-Ha-ras-transformed NIH 3T3 cells (RS485 cell line) with suramin leads to the induction of lysyl oxidase (Palamakumbura et al., 2003). Here we investigated the effects of suramin on the rate of growth and morphology of RS485 cells, as an initial measure of cell phenotype. Cells were plated in 6-well plates and cultured for 24 hours and then grown in the continuous presence of 0, 100, 125, or 150 μM suramin. As control, the growth of phenotypically normal NIH 3T3 cells in the absence of suramin was analyzed at the same time. Cell growth was determined by daily crystal violet staining of replicate wells. RS485 cells grew more rapidly than NIH 3T3 cells, as expected (Chang et al., 1982). Suramin significantly decreased the growth rate in a dose-dependent manner. Data show that 100, 125 and 150 μM suramin decreased the growth rate by 38%, 49%, and 56%, respectively, calculated from linear regression analyses of log of absorbance vs time. The growth rate of RS485 cells treated with 150 μM suramin was similar to that of NIH 3T3 cells. Furthermore, treatment with suramin caused a dose-dependent change in the morphology of RS485 cells with cells appearing less transformed, i.e., flatter and contact inhibited in the presence of 150 μM suramin.

We next determined the effects of suramin on cell cycle progression of RS485 cell cultures. As shown in Table I, fluorescence activated cell sorting (FACS) indicated that treatment with 100 or 150 μM suramin increased the percentage of RS485 cells in G₁ and decreased that in S phase in a dose-dependent manner. The proportions of cells in G₁ and S phases in cultures treated with 150 μM suramin are nearly identical to untreated phenotypically normal NIH 3T3 cells (Table I). Thus, treatment with 150 μM suramin changed the morphology, cell cycle, and growth rate of RS485 cells resulting in a more normal phenotype.

Inhibition of lysyl oxidase expression reduces phenotypic reversion induced by suramin. The previous experiment showed that suramin causes phenotypic changes in RS485 cells. To determine that the suramin-induced phenotypic changes depend upon lysyl oxidase expression, we generated stable anti-sense lysyl oxidase transfected RS485 cell clones. Nine anti-sense lysyl oxidase transfected clones and six empty vector transfected clones were generated and grown in the presence and absence of 150 μM suramin, and after 24 hours, cells were fixed and subjected to cell cycle analysis. Non-transfected RS485 cells were analyzed as an additional control. Differences in the percentage of cells in G₁ and S phase occurred as a function of suramin treatment. In empty vector transfected clones, suramin increased the average number of cells in G₁ by 20.8%, and decreased the average number of cells in S phase by 13.5%. As expected, these values are not significantly different from non-transfected cells. In contrast, anti-sense lysyl oxidase transfected clones show only a 10.6% increase in the percentage of cells in G₁ phase and a 6.9% decrease in the S phase after suramin treatment. These changes are significantly smaller than either of the control groups of cells.

To confirm that lysyl oxidase expression is actually diminished by anti-sense lysyl oxidase transfection, the ability of 150 μM suramin to induce low steady state mRNA levels of lysyl oxidase in clones was assessed after 24 hours of treatment by Northern blot analysis with normalization to 18S rRNA signals. Suramin treatment led to an average 8-fold increase in lysyl oxidase mRNA levels in the six empty vector-transfected clones assayed, consistent with previous studies on non-transfected RS485 cells (Palamakumbura et al., 2003). In contrast, an average 1.8-fold increase in lysyl oxidase mRNA levels occurred in the nine anti-sense lysyl oxidase transfected clones, as shown by Northern blot analysis. Thus, the induction of steady state lysyl oxidase mRNA levels by suramin is inhibited by the anti-sense lysyl oxidase transfection, as expected. Taken together, these data demonstrate that anti-sense lysyl oxidase transfected cells have significantly diminished suramin-induced cell cycle changes compared to those of empty-vector transfected or non-transfected RS485 cells. These data indicate that lysyl oxidase expression plays a role in mediating the phenotypic effects of suramin on RS485 cells.

BAPN-mediated inhibition of lysyl oxidase enzyme activity fails to prevent phenotypic reversion induced by suramin. It has generally been assumed, although never directly tested, that lysyl oxidase dependent phenotypic reversion and tumor suppressor activity depends on its enzyme activity. To directly test the role of lysyl oxidase enzyme activity, we measured the effects of the lysyl oxidase inhibitor, BAPN (Tang et al., 1983), on the phenotypic changes in RS485 cells following treatment with 150 μM suramin. We have previously shown that suramin induces lysyl oxidase activity by about 2.5-fold (Palamakumbura et al., 2003). RS485 cell growth was assessed in the absence or presence of either 150 μM suramin or 400 μM BAPN, or a combination of both 150 μM suramin and 400 μM BAPN. This concentration of BAPN effectively inhibits lysyl oxidase (Kagan et al., 1982). In contrast, suramin decreased the growth rate of RS485 cells whereas BAPN had no effect on the growth rate. Surprisingly, BAPN did not reverse or affect in any detectable way the suramin-mediated inhibition of RS485 cell growth.

Studies performed with the stable phenotypic revertant cell line PR4 demonstrated that lysyl oxidase expression specifically is required to maintain the normal phenotype (Contente et al., 1990; Kenyon et al., 1991). These studies utilized anti-sense transfection methodology to reduce lysyl oxidase expression resulting in transformation, but did not directly investigate the role of lysyl oxidase enzyme activity in phenotypic reversion. If lysyl oxidase enzyme activity were required for the normal phenotype of PR4 cells, then BAPN would cause re-transformation. Thus, growth curves were generated for PR4 cells in the presence of 0 or 400 μM BAPN. BAPN did not affect the growth rate of PR4 cells. Furthermore, BAPN did not change the morphology of PR4 cells. Assays of PR4 cell culture media confirmed that cells grown without BAPN produce easily detectable lysyl oxidase enzyme activity (14,000+/−400 dpm/×10⁶ cells), whereas no lysyl oxidase enzyme activity was detected in the medium of cultures grown at the same time in the continuous presence of both 200 μM and 400 μM BAPN using a highly sensitive assay for lysyl oxidase enzyme activity (Bedell-Hogan et al., 1993). Taken together, these findings show that growth inhibition of RS485 cells by suramin does not depend on lysyl oxidase enzyme activity. Similarly, inhibition of lysyl oxidase enzyme activity does not affect growth of stable phenotypic revertants that require lysyl oxidase expression for the normal cell phenotype.

Lysyl oxidase pro-peptide and not the active enzyme causes phenotypic reversion of anti-sense lysyl oxidase transfected PR4 cells (AS-3B) and RS485 cells.

The biosynthesis of lysyl oxidase includes extracellular proteolysis of 48-50 kDa pro-lysyl oxidase by procollagen C-proteinases to release the 30 kDa lysyl oxidase enzyme and an 18 kDa cationic pro-peptide. The question, therefore, arises as to whether the released pro-peptide contributes to phenotypic reversion. To determine the effect of recombinant rat lysyl oxidase pro-peptide on the phenotype, we first chose to study the effect of the lysyl oxidase pro-peptide on cell cycle parameters of AS-3B and RS485 transformed cell lines. AS-3B cells are PR4 cells transformed by stable transfection with anti-sense lysyl oxidase expression vector (Contente et al., 1990; Kenyon et al., 1991) and should be sensitive to features of lysyl oxidase that cause phenotypic reversion. Cells were plated on 6-well plates that had been coated with 0, 1, 5, or 10 μg pro-peptide per well. This experimental approach was taken due to the poor solubility of the pro-peptide in cell culture media and physiologic buffers. After 4 days, cells (1−2×10⁶) were harvested and subjected to cell cycle analysis. For AS-3B cells (Table II) the percentage of cells in G₁ phase increased in the presence of the pro-peptide by about 6% while the percentage of cells in S phase decreased by 5.7%, suggesting that the lysyl oxidase pro-peptide has a role in altering the cell cycle of AS-3B cells. Similarly, in RS485 cells (Table III) the percentage of cells in G₁ phase increased by 7.6% in the presence of the pro-peptide, with a corresponding decrease in the percentage of cells in S phase. Results suggest that pro-peptide affects cell cycle progression of both AS-3B and RS485 cells.

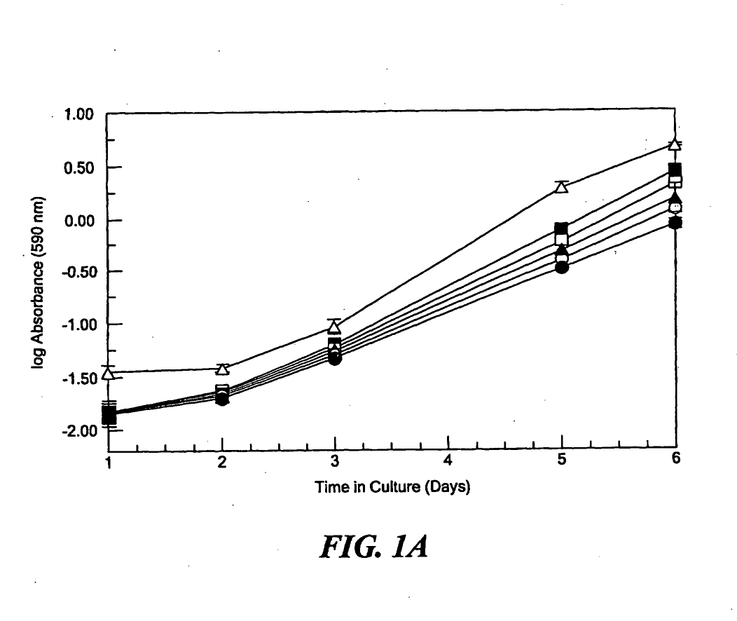

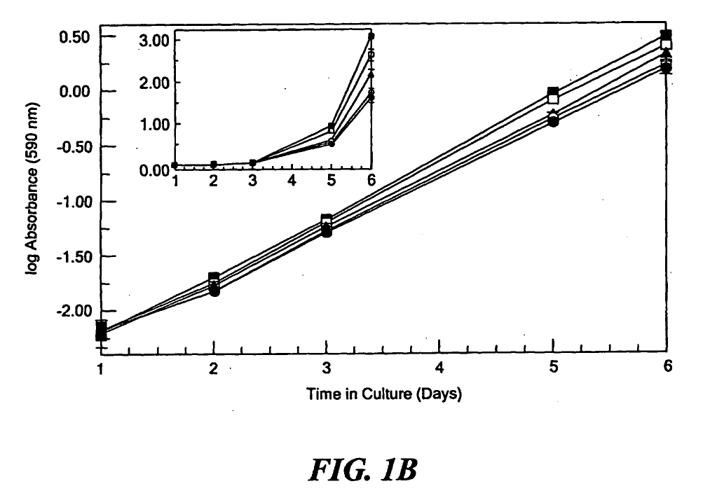

We next investigated the effects of the lysyl oxidase pro-peptide and of the mature 30 kDa enzyme on the growth of AS-3B and RS485 cells using coated 24-well plates. As shown in FIGS. 1A and 1B, pro-peptide decreased the growth of both cell lines in a dose-dependent manner. Linear regression analyses of plots of the log of absorbance vs time demonstrated dose-dependent growth inhibition of 6.0%, 9.3%, 13.6% and 17.6% for AS-3B cells and 4.1%, 5.6%, 8.9%, and 11.9% for RS485 cells with 0.2, 1, 2 or 4 μg of lysyl oxidase pro-peptide, respectively. Moreover, the lysyl oxidase pro-peptide did not affect the plating efficiency of these cells, as initial crystal violet absorbance values were essentially identical for cells plated on pro-peptide compared to no pro-peptide. No obvious effect of pro-peptide on cell morphology was observed. As an additional control in FIG. 1A, AS-3B cells were grown at the same time on mature 30 kDa lysyl oxidase enzyme. In contrast to the effects of the lysyl oxidase pro-peptide, lysyl oxidase enzyme did not inhibit the growth rate of AS-3B cells (FIG. 1A). In fact, growth on lysyl oxidase enzyme appeared to be slightly increased compared to the control. As seen in FIG. 1A, this is due to higher plating efficiency of cells on the mature enzyme. Linear regression analyses showed that the rate of cell growth on lysyl oxidase enzyme was essentially unaffected (increased by 2%), even though plating efficiency was increased. Taken together, the data indicate that the lysyl oxidase pro-peptide has a specific inhibitory effect on cell growth and cell cycle progression that contributes to phenotypic reversion.

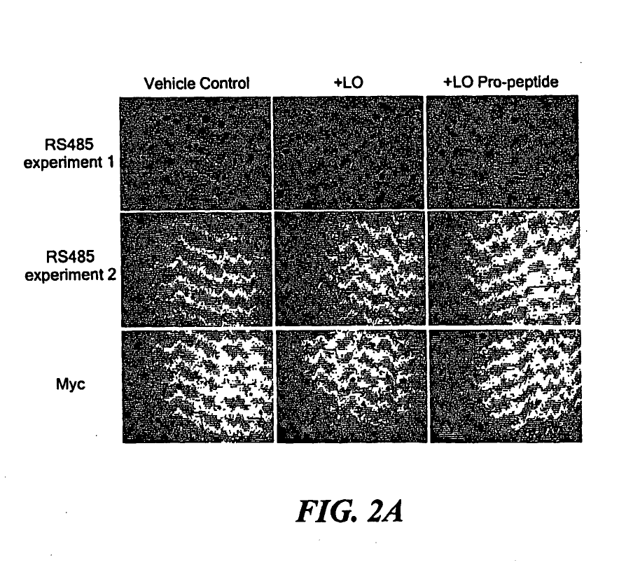

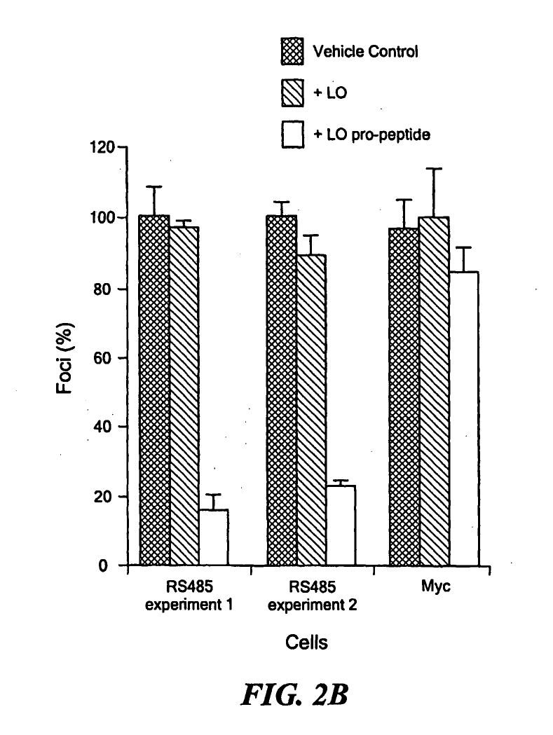

Lysyl oxidase pro-peptide, and not the active enzyme, inhibits growth of RS485 cells in soft agar. A hallmark of transformed cells is the ability to grow in soft agar and to form colonies, whereas non-transformed cells are unable to grow when suspended in soft agar. The respective effects of active 30 kDa lysyl oxidase enzyme, and of the 18 kDa lysyl oxidase pro-peptide vs vehicle control on the ability of RS485 cells to grow in soft agar were determined. Lysyl oxidase pro-peptide was strongly inhibitory, whereas the 30 kDa lysyl oxidase enzyme was unable to inhibit growth of RS485 cells in soft agar (FIG. 2). In two separate experiments an average 80% reduction in colony formation was observed. Lysyl oxidase mediated reversion appears to be selective for ras-transformed cells (Contente et al., 1990; Kenyon et al., 1991). To investigate the specificity of the propeptide, its growth inhibitory on c-myc-transformed M158 fibroblasts was determined. Neither lysyl oxidase pro-peptide nor the 30 kDa lysyl oxidase enzyme inhibited the growth in soft agar of c-myc transformed M158 fibroblasts (FIG. 2). Thus, the 18 kDa lysyl oxidase pro-peptide, and not the active lysyl oxidase enzyme, inhibits ras-dependent transformation.

### Materials and Methods

Chemicals. Suramin was either kindly provided by the Division of Cancer Treatment Diagnosis and Centers, National Cancer Institute and Parke-Davis, or was purchased from Sigma (St Louis, Mo.). All other chemicals and reagents were purchased from Sigma or Gibco BRL (Rockville, Md.).

Cell culture. RS485 cells are transformed by over-expression of c-Ha-ras in NIH 3T3 cells (Chang et al., 1982). PR4 cells are stable phenotypic revertants of RS485 cells obtained after treatment with a/p interferon (Samid et al., 1987), and AS-3B cells are re-transformed after transfection of PR4 cells with anti-sense lysyl oxidase (Contente et al., 1990; Kenyon et al., 1991). Cells were plated onto 100 mm cell culture plates in Dulbecco's modified Eagle's medium, containing 10% fetal bovine serum (FBS) plus 1% nonessential amino acids, 100 units/ml penicillin and 100 μg/ml streptomycin. Cultures were maintained at 37° C. in a fully humidified atmosphere of 5% CO₂ in air. Cells in logarithmic growth phase, were dissociated with trypsin/EDTA, and inoculated at a desired density for each experiment.

Growth curves. To study cell growth rates, cells were plated in 6-well plates at a density of 35,000 cells/well and were grown in complete medium containing 10% FBS, as described above. Additions of suramin or β-aminopropionitrile (BAPN), when appropriate, were initiated 24 hours after plating. Media were changed every three days in the continuous presence of suramin or BAPN, as indicated for each experimental design in the Results section. Cell density was determined in triplicate every day by crystal violet staining, as described (Kueng et al., 1989; Gillies et al., 1986). Cells were fixed with 10% formalin in PBS, washed with PBS, and then stained for 30 minutes with 0.1% crystal violet at room temperature with shaking. Unbound dye was then removed by washing with water until washes were colorless. Bound dye was then eluted with 10% acetic acid, and quantitated by measuring the absorbance at 590 nm. For quantitative analyses of growth rates, the logarithmic value of absorbance vs time was plotted±standard deviation and the rates were calculated by linear regression analyses. In addition, data were plotted as total absorbance +/−SE vs time. Experiments were performed three times each with consistent findings.

Lysyl oxidase enzyme activity. PR4 cells were plated in 100 mm cell culture plates and then grown and re-fed every two days as described above in the constant presence of 0, 200 and 400 μM BAPN for seven days until visually confluent. Cells were then re-fed with serum-free medium supplemented with 0.1% bovine serum albumin still in the constant presence or absence of BAPN. After 24 hours conditioned 0.3 ml aliquots of media samples were assayed in quadruplicate using a tritiated recombinant human tropoelastin as substrate as previously described (Bedell-Hogan et al., 1993). Incubations were performed at 0.37° C. for 90 minutes (Hong et al., 1999) and data were expressed as total cpm released +/−SE per culture.

Cell cycle analysis. RS485, NIH 3T3 and PR4 cells were plated on 100 mm plates and were grown until confluent with 0 or 150 μM suramin. Cells (1.5−2×10⁶) were then trypsinized, washed with PBS and fixed by washing with ice-cold 70% ethanol. Cells were stained with propidium iodide (50 μg/ml) in PBS containing 2% FBS and was analyzed by flow cytometry using a FACScan flow cytometry with CELLQUEST acquisition and analysis software (Becton Dickinson Co, Bedford, Mass.).

RNA isolation and Northern blot analysis. Total RNA was isolated using the RNeasy —RNA isolation kit (Qiagen, Valencia, Calif.). Ten μg samples of denatured RNA were electrophoresed on a 1% agarose gel containing 18% formaldehyde. Gels were transferred in 10×SSC by capillary blotting overnight to Gene Screen nylon membranes (Perkin Elmer Life Science, Boston, Mass.). The membranes were hybridized at 42° C. as previously described (Feres-Filho et al., 1995) with labeled mouse lysyl oxidase probe (Contente et al., 1990; Kenyon et al., 1991), prepared by random primer labeling (Feinberg et al., 1983). For normalization and as a measure of constant loading of gels, blots were stripped and rehybridized with a radiolabeled 18S rRNA probe (Hillis et al., 1991). Autoradiograms were assessed and normalized by densitometric scanning on a Versa Doc Model 3000 Gel Documentation System and Quantity One Software (BioRad, Hercules, Calif.).

Stable transfection of RS485 cells with an anti-sense lysyl oxidase expression vector. Cells were grown in 100 mm cell culture dishes. At approximately 70% confluence, they were transfected with the anti-sense murine lysyl oxidase expression vector pCLO3 (Giampuzzi et al., 2001) (20 μg DNA/plate) using the calcium phosphate precipitation method (Graham et al., 1973). As a control, RS485 cells were transfected with empty vector (pcDNA3). The transfected cells were selected using G418 (geneticin) at a final concentration of 400 μg/ml in the medium. Colonies were isolated from anti-sense lysyl oxidase and empty vector transfected RS485 cells using cloning cylinders (Southern et al., 1982) and cultured in 400 μg/ml G418. Cells were then plated onto 100 mm plates (250,000 cells/plate) without G418 and treated with 0 or 150 μM suramin. After 24 hours, cells were prepared for cell cycle and Northern analysis as described above.

Lysyl oxidase pro-peptide coated cell culture plates. Rat lysyl oxidase pro-peptide was expressed in E. coli and purified as described (Hong et al., 2004). The pro-peptide (200-400 μg/ml) was then dialyzed against 16 mM phosphate buffer, pH 7.8 for 5 hours and 6-well plates were coated with 0, 1, 5, or 10 μg pro-peptide in 1 ml water per well and left overnight under UV light in the cell culture hood to completely dry. AS-3B or RS485 cells were then plated at a density of 35,000 cells per well on the pro-peptide-coated plates and cultured until visual confluence. Cells were then prepared for cell cycle analysis as described above. To study the effect of pro-peptide on the growth rate of AS-3B and RS485 cells, 24-well plates were coated with 0, 0.2, 1, 2, or 4 μg pro-peptide in 350 μl water per well, as above, and the cells plated at a density of 7,000 cells/well. Cell density was determined in triplicate every day, by crystal violet staining as described above. In selected experiments, mature 30 kDa lysyl oxidase (Feinberg et al., 1983) was dialyzed against 16 mM potassium phosphate buffer, pH 7.8 for 5 hours, and 4 μg coated in 24 well plates at the same time as described for the pro-peptide.

Focus formation assay in soft agar. RS485 cells and myc-transformed M158 cells were plated, in duplicate, at 10⁴ cells/ml in top plugs consisting of complete Ham F-12 nutrient mixture medium and 0.4% SeaPlaque agarose (FMC Bioproducts, Rockland, Me.) in the presence of 2.5 μg purified bovine aorta lysyl oxidase enzyme (30 kDa form) (Palamakumbura et al., 2002), or with 2.5 μg recombinant rat lysyl oxidase pro-peptide (18 kDa form) (Hong et al., 2004), or the same volume of vehicle potassium phosphate (16 mM, pH 7.8). After 2 weeks incubation in a humidified incubator at 37° C., the colonies were stained with 0.5 ml of 0.0005% crystal violet and photographed using a digital camera coupled to a dissection microscope (×50 magnification). Three random fields were counted from each of two duplicate samples, and average values presented±SD.

Transient transfections, luciferase assays and fluorescence microscopy. NIH 3T3 and RS485 cells were plated in 35 mm culture dishes. Cells were transfected overnight, in triplicate, with the indicated expression vectors by using Fugene 6 transfection reagent (Roche Molecular Biochemicals, Indianapolis, Ind.) in Dulbecco's Modified Eagles Medium containing 0.5% FBS. The plasmids used are: pCMVneo-Myr-Akt kindly provided by Z. Luo (Boston University Medical School, Boston, Mass.), NF-κB-luciferase which was a gift from G. Rawadi (Hoechst-Marion-Roussel, Romainville, France), pEGFP-C1-PDK1 kindly provided by J. Chung (Korean Advanced Institute of science and Technology, Taejon, Republic of Korea), and pcDNA3.1 (+)/LOPP propeptide and pcDNA4-LO enzyme expression vectors. The expression vector for the lysyl oxidase pro-peptide pcDNA3.1 (+)/LOPP was generated from pSV40 PolyACOD (Trackman et al., 1992) by PCR, using forward primer: 5′-AC TGGATCCCGA AGAGGTCTCC CTCCTTCGCG-3′ and reverse primer 5′-TACGAAT TCTCAGCCCA CCATGCGATC TACGTGGCTG-3′. The DNA was digested with BamHI and EcoRI and gel purified and cloned into pcDNA3.1 (+) (Invitrogen), resulting in pcDNA3.1 (+)/LOPP. This construct contains the rat cDNA sequence (−94 to +486) that includes a portion of the 5″-UTR, the signal peptide, the entire rat lysyl oxidase propeptide coding region and no mature lysyl oxidase sequence. The insert was directly confirmed by DNA sequencing. The expression vector for mature lysyl oxidase was accomplished by excision of nucleotides encoding amino acid residues 23-157 from a construct of murine lysyl oxidase cDNA −33 to +1234, and then cloned into pcDNA4 as previously reported (Seve et al., 2002).

For luciferase assays, 1 □g NF-κB-dependent luciferase reporter plasmid and 0.5 μg pSV40-β-Gal reporter gene were co-transfected with the indicated DNAS. Cells were stimulated with addition of FBS to a final concentration of 10%, and total cell extracts were prepared after 48 hours. The resulting extracts were normalized for β-Gal expression and used in a luciferase activity assay, according to the manufacturer's instructions (Promega kit). Results are expressed as the fold induction of luciferase activity calculated under stimulation conditions (10% FBS) compared to starvation conditions (0.5% FBS). The results are expressed as the mean ±the standard deviation (SD). For fluorescence microscopy, cells were co-transfected with a vector expressing green fluorescent protein-tagged EDKL protein (GFP-PDK1), and either lysyl oxidase propeptide or lysyl oxidase enzyme expression vectors or parental empty vectors for 48 hours. Localization of the GFP-PDK1 was determined using an Anxiovert 200 M fluorescent microscope (Carl Zeiss MicroImaging, Inc., Thornwood, N.Y.). Analyses and pictures were performed by using Axiovision (v.3.1 software; Carl Zeiss MicroImaging, Inc.).

### REFERENCES

Bedell-Hogan, D., Trackman, P., Abrams, W., Rosenbloom, J., and Kagan, H. (1993) J Biol Chem 268, 10345-10350.
Belting, M. (2003) Trends Biochem Sci 28, 145-151.
Cantrell, D. A. (2002) Nat Rev Immunol 2, 20-27.
Chang, E. H., Furth, M. E., Scolnick, E. M., and Lowy, D. R. (1982) Nature 297, 479-48.
Coffey, R. J., Jr., Leof, E. B., Shipley, G. D., and Moses, H. L. (1987) J Cell Physiol 132, 143-148.
Contente, S., Kenyon, K., Rimoldi, D., and Friedman, R. M. (1990) Science 249, 796-798.
Cvejic, S., Jiang, Y., Huang, X. (2000) Trends Cardiovasc Med. 10, 160-165.
Feinberg, A. P., and Vogelstein, B. (1983) Anal Biochem 132, 6-13.
Feres-Filho, E. J., Choi, Y. J., Han, X., Takala, T. E., and Trackman, P. C. (1995) J Biol Chem 270, 30797-30803.
Fukumoto, M., Takahashi, J. A., Murai, N., Ueba, T., Kono, K., and Nakatsu, S. (2000) Anticancer Res 20, 4059-4065.
Garcia-Schurmann, J. M., Schulze, H., Haupt, G., Pastor, J., Allolio, B., and Senge, T. (1999) Urology 53, 535-541.
Giampuzzi, M., Botti, G., Cilli, M., Gusmano, R., Borel, A., Sommer, P., and Di Donato, A. (2001) J Biol Chem 276, 29226-29232.
Gillies, R. J., Didier, N., and Denton, M. (1986) Anal Biochem 159, 109-113.
Graham, F. L., and van der Eb, A. J. (1973) Virology 52, 456-467.
Hajnal, A., Klemenz, R., and Schafer, R. (1993) Cancer Res 53, 670-4675.
Hamalainen, E. R., Kemppainen, R., Kuivaniemi, H., Tromp, G., Vaheri, A., Pihlajaniemi, T., and Kivirikko, K. I. (1995) J Biol Chem 270, 21590-21593.
Hendry, B. M., Sharpe, C. C. (2003) Nephron Exp Nephrol 93, e129-33.
Hillis, D. M., and Dixon, M. T. (1991) Q Rev Biol 66, 411-453. Hong, H. H., Pischon, N., Santana, R. B., Palamakumbura, A. H., Babakhanlou-Chase, H., Gantz, D., Guo, Y., Uzel, M. I., Ma, D., and Trackman, P. C. (2004) J. Cellular Physiol. 20, 53-62.
Hong, H. H., Uzel, M. I., Duan, C., Sheff, M. C., and Trackman, P. C. (1999) Lab Invest 79, 1655-1667.
Kagan, H. M. (1986) in Biology and Regulation of Extracellular Matrix: A Series. Regulation of Matrix Accumulation. (Mecham, R. P., ed) Vol. 1, pp. 321-398, Academic Press, Orlando.
Kagan, H. M., and Li, W. (2003) J Cell Biochem 88, 660-672.
Kagan, H. M., and Trackman, P. C. (1991) Am J Respir Cell Mol Biol 5, 206-210.
Kenyon, K., Contente, S., Trackman, P. C., Tang, J., Kagan, H. M., and Friedman, R. M. (1991) Science 253, 802.
Kessler, E., Takahara, K., Biniaminov, L., Brusel, M., and Greenspan, D. S. (1996) Science 271, 360-362.
Krzyzosiak, W. J., Shindo-Okada, N., Teshima, H., Nakajima, K., and Nishimura, S. (1992) Proc Natl Acad Sci USA 89, 4879-4883.
Kueng, W., Silber, E., and Eppenberger, U. (1989) Anal Biochem 182, 16-19.
Kuivaniemi, H., Korhonen, R. M., Vaheri, A., and Kivirikko, K. I. (1986) FEBS Lett 195, 261-264.
La Rocca, R. V., Stein, C. A., Danesi, R., Jamis-Dow, C. A., Weiss, G. H., and Myers, C. E. (1990a) J Clin Endocrinol Metab 71, 497-504.
La Rocca, R. V., Stein, C. A., Danesi, R., and Myers, C. E. (1990b) J Steroid Biochem Mol Biol 37, 893-898.
Mariani, T. J., Trackman, P. C., Kagan, H. M., Eddy, R. L., Shows, T. B., Boyd, C. D., and Deak, S. B. (1992) Matrix 12, 242-248.
Mietz, H., Chevez-Barrios, P., Feldman, R. M., and Lieberman, M. W. (1998) Br J Opthalmol 82, 816-820.
Molkentin, J. D., Dorn, II, G. W. 2^{nd} (2001) Annu Rev Physiol. 63, 391-426.
Nellaiappan, K., Risitano, A., Liu, G., Nicklas, G., and Kagan, H. M. (2000) J Cell Biochem 79, 576-582.
Palamakumbura, A. H., Sommer, P., and Trackman, P. C. (2003) J Biol Chem 278, 30781-30787.
Palamakumbura, A. H., and Trackman, P. C. (2002) Anal Biochem 300, 245-251.
Panchenko, M. V., Stetler-Stevenson, W. G., Trubetskoy, O. V., Gacheru, S, N., and Kagan, H. M. (1996) J Biol Chem 271, 7113-7119.
Ren, C., Yang, G., Timme, T. L., Wheeler, T. M., and Thompson, T. C. (1998) Cancer Res 58, 1285-1290.
Samid, D., Flessate, D. M., and Friedman, R. M. (1987) Mol Cell Biol 7, 2196-2200.
Schwartz, R. H. (2003) Annu Rev Immunol 21, 305-34.
Seve, S., Decitre, M., Gleyzal, C., Farjanel, J., Sergeant, A., Ricard-Blum, S., and Sommer, P. (2002) Connect Tissue Res 43, 613-619.
Small, E. J., Meyer, M., Marshall, M. E., Reyno, L. M., Meyers, F. J., Natale, R. B., Lenehan, P. F., Chen, L., Slichenmyer, W. J., and Eisenberger, M. (2000) J Clin Oncol 18, 1440-1450.
Song, S., Wientjes, M. G., Walsh, C., and Au, J. L. (2001) Cancer Res 61, 6145-6150.
Southern, P. J., and Berg, P. (1982) J Mol Appl Genet. 1, 327-341.
Sullivan, K. A., Kim, B., Buzdon, M., and Feldman, E. L. (1997) Brain Res 744, 199-206.
Tang, S. S., Trackman, P. C., and Kagan, H. M. (1983) J Biol Chem 258, 4331-4338.
Trackman, P. C., Bedell-Hogan, D., Tang, J., and Kagan, H. M. (1992) J Biol Chem 267, 8666-8671.
Trackman, P. C., Pratt, A. M., Wolanski, A., Tang, S. S., Offner, G. D., Troxler, R. F., and Kagan, H. M. (1990) Biochemistry 29, 4863-4870.
Uzel, M. I., Scott, I. C., Babakhanlou-Chase, H., Palamakumbura, A. H., Pappano, W. N., Hong, H. H., Greenspan, D. S., and Trackman, P. C. (2001) J Biol Chem 276, 22537-22543.
Wong, C. K., Zhang, J., Ip, W. K., Lam, C. W. (2002) immunopharmacol Immunotoxicol 24, 165-86.
Zanghi, J. A., Renner, W. A., Bailey, J. E., and Fussenegger, M. (2000) Biotechnol Prog 16, 319-325.

TABLE I
Effects of Suramin on the Cell Cycle Progression odf RS485 Cells

| Cell Type | G1 % | S % | G2, M % |
|---|---|---|---|
| RS485 | 43.3 ± 3.6 | 30.8 ± 4.6 | 25.8 ± 1.1 |
| RS485 with 100 μM suramin | 59.4 ± 1.3 | 21.5 ± 2.4 | 19.1 ± 7.9 |
| RS485 with 150 μM suramin | 67.4 ± 0.2 | 19.0 ± 1.7 | 13.6 ± 1.9 |
| NIH 3T3 | 66.2 ± 2.1 | 17.4 ± 0.4 | 16.4 ± 3.0 |

RS485 cells were grown in the presence of 0, 100, or 150 μM suramin for 2 days. Alternatively, NIH 3T3 cells were grown in the absence of suramin, as control. Cells (1.5−2×10⁶) were fixed with 70% ethanol and stained with propidium iodide and analyzed by flow cytometry. Data shown are the averages ±SD of experiments performed three times.

TABLE II
Effects of the Lysyl Oxidase Pro-peptide on Cell Cycle
Progression of AS-3B Cells

| Cell Type | G1 % | S % | G2, M % |
|---|---|---|---|
| AS-3B | 74.2 ± 1.0 | 14.9 ± 1.2 | 10.9 ± 0.8 |
| AS-3B with 1 μg pro-peptide | 74.7 ± 0.9 | 12.2 ± 0.5 | 13.1 ± 1.2 |
| AS-3B with 5 μg pro-peptide | 77.6 ± 2.0 | 10.61 ± 1.7 | 1.8 ± 1.6 |
| AS-3B with 10 μg pro-peptide | 79.9 ± 1.3 | 9.29 ± 1.3 | 10.9 ± 0.5 |

AS-3B cells were grown on 0, 1, 5, or 10 μg of pro-peptide coated 6-well plates and cultured for 4 days. Cells (1.5−2×10⁶) were fixed with 70% ethanol and stained with propidium iodide and analyzed by flow cytometry. Data shown are the averages ±SD of experiments performed three times.

TABLE III
Effects of the Lysyl Oxidase Pro-peptide on Cell Cycle
Progression of RS485 Cells

| Cell Type | G1 % | S % | G2, M % |
|---|---|---|---|
| RS485 | 53.5 ± 0.7 | 22.6 ± 0.8 | 23.8 ± 1.6 |
| RS485 with 1 μg pro-peptide | 57.3 ± 1.7 | 20.8 ± 2.3 | 21.2 ± 1.9 |
| RS485 with 5 μg pro-peptide | 59.2 ± 2.3 | 19.1 ± 1.6 | 21.7 ± 2.1 |
| RS485 with 10 μg pro-peptide | 61.1 ± 2.4 | 14.9 ± 0.5 | 23.9 ± 1.9 |

RS485 cells were grown on 0, 1, 5, or 10 μg of pro-peptide coated 6-well plates and cultured for 2 days. Cells (1.5−2×10⁶) were fixed with 70% ethanol and stained with propidium iodide and analyzed by flow cytometry. Data shown are the averages ±SD of experiments performed three times.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A therapeutic composition comprising a polypeptide comprising a therapeutically active portion of lysyl oxidase pro-peptide, said polypeptide being in a pharmaceutically acceptable carrier substance therefore, wherein said polypeptide does not have lysyl oxidase enzymatic activity.

2. The therapeutic composition of claim 1, wherein said polypeptide is active in inhibiting cell growth in soft agar.

3. The therapeutic composition of claim 1, wherein said polypeptide is active in inhibiting tumor formation.

4. The therapeutic composition of claim 1, comprising a polypeptide comprising an active portion of the amino acid sequence given in SEQ ID NO.: 1 or SEQ ID NO.: 2, or conservative substitions thereof.

5. The therapeutic composition of claim 1, comprising a polypeptide comprising an active portion of an amino acid sequence selected from the group consisting of SEQ ID NOs.: 3-8, or conservative substitions thereof.

6. A method of identifying the active portion of lysyl oxidase pro-peptide, said method comprising the steps of:
a) providing cells transformed with an oncogene, wherein growth of said transformed cells is known to be inhibited by lysyl oxidase pro-peptide;
b) culturing said cells in the presence of a fragment of lysyl oxidase pro-peptide of length l₁, wherein said l₁ fragment is known to comprise said active portion of lysyl oxidase pro-peptide;
c) determining a value for the effectiveness of said l₁ fragment in inhibiting growth of said transformed cells in soft agar;
d) culturing another aliquot of said cells with a smaller portion of said lysyl oxidase pro-peptide, of length l₂;
e) determining a value for the effectiveness of said l₂ fragment in inhibiting growth of said transformed cells in soft agar; and
f) repeating steps d) and e) with progressively smaller portions of said lysyl oxidase pro-peptide, of length lᵢ, until the minimum sized active portion is determined.

7. The method of claim 6, wherein said transformed cells are cultured in soft agar.

8. A method of treating a patient, said method comprising the steps of:
providing a patient suffering from cancer; and
administering to said patient a therapeutically effective amount of the composition of claim 1.

9. The method of claim 8, wherein said patient suffers from a form of cancer dependent on ras signaling for cell transformation.

10. A method of treating a patient, said method comprising the steps of:
providing a patient suffering from a disease or disorder that occurs via elevated ras-dependent signaling; and
administering to said patient a therapeutically effective amount of the composition of claim 1.

11. The method of claim 9, wherein said patient suffers from colon, breast, lung or prostate cancer.

12. The method of claim 10, wherein said disease or disorder is selected from the group consisting of diseases or disorders of the kidney, cardiovascular system and immune system.

13. The method of claim 10, wherein said patient suffers from a bone disease.

14. The method of claim 13, wherein said bone disease is an osteopenic condition.

15. The method of claim 14, wherein said bone disease is osteoporosis.


SEQUENCE LISTING (8 sequences)

SEQ ID NO: 1 - protein, 147 aa, Human
  PROPEP (22)...(168)
APPAAGQQQPPREPPAAPGAWRQQIQWENNGQVFSLLSLGSQYQPQRRRDPGAAVPGAANASAQQPRTPILLIRDNRTAAARTRTAGSSGVTAGRPRPTARHWFQAGYSTSRAREAGASRAENQTAPGEVPALSNLRPPSRVDGMVG

SEQ ID NO: 2 - protein, 141 aa, Mouse
  PROPEP (22)...(162)
APQTPREPPAAPGAWRQTIQWENNGQVFSLLSLGAQYQPGRRRDPSATARRPDGDAASQPRTPILLLRDNRTASTRARTPSPSGVAAGRPRPAARHWFQAGFSPSGARDGASRRAANRTASPQPPQLSNLRPPSHIDRMVG

SEQ ID NO: 3 - protein, 35 aa, Human
  PROPEP (32)...(66)
PREPPAAQGAWRQQIQWENNGQVFSLLSLGSQYQP

SEQ ID NO: 4 - protein, 35 aa, Mouse
  PROPEP (26)...(60)
PREPPAAPGAWRQTIGWENNGQVFSLLSLGAQYQP

SEQ ID NO: 5 - protein, 35 aa, Rat
  PROPEP (26)...(60)
PREPPAAPGAWRQTIQWENNGQVFSLLSLGAQYQP

SEQ ID NO: 6 - protein, 38 aa, Human
  PROPEP (84)...(121)
AQQPRTPILLIRDNRTAAARTRTAGSSGVTAGRPRPTA

SEQ ID NO: 7 - protein, 38 aa, Mouse
  PROPEP (78)...(115)
ASQPRTPILLLRDNRTASTRARTPSPSGVAAGRPRPAA

SEQ ID NO: 8 - protein, 38 aa, Rat
  PROPEP (78)...(115)
AAQPRTPILLLRDNRTASARARTPSPSGVAAGRPRPAA